(19) United States
(12) Patent Application Publication
(10) Pub. No.: US 2012/0135962 A1
(43) Pub. Date: May 31, 2012

(54) COMPOSITIONS TO COMBAT ECTOPARASITES

(75) Inventor: Baard Johannessen, Arendal (NO)

(73) Assignee: NETTFORSK AS, Arendal (NO)

(21) Appl. No.: 13/259,783

(22) PCT Filed: Mar. 25, 2010
(86) PCT No.: PCT/GB2010/000569

§ 371 Date: Dec. 6, 2011

(30) Foreign Application Priority Data
Mar. 25, 2009 (GB) 0905165.7

(51) Int. Cl. A61K 31/675; A61P 33/14

(52) U.S. Cl. 514/81

(57) ABSTRACT

The present invention provides a method of pesticidal treatment of vertebrate aquatic animals, in particular fish. The method includes applying to said aquatic animals, typically topically or, more preferably, by administration into water in which said aquatic animals are contained, a topical pesticidal composition comprising a pyrethroid and an organophosphate biocide in a weight ratio of 1:20 to 10:1, particularly 1:10 to 5:1, especially 1:3 to 3:1.

The present invention relates to biocidal compositions for treatment to combat infestation by exoskeletal ectoparasite species, in particular lice, insects, arachnids, etc. and to a method of treatment using such compositions.

In agriculture and aquaculture, the crop is frequently plagued by exoskeletal pests. For example, in aquaculture sea lice, in plant crops aphids and various insects, and in land animals fleas and lice. Likewise, humans may be infested by head lice and other exoskeletal pests.

Ectoparasites of the orders Phthiraptera (lice), Acarina (mites) and Siphonaptera (fleas), more especially Pediculus humanus capitis (human head lice), body louse (Pediculus humanus humanus), the pubic louse (Pthirus pubis) and the scabies mite (Sarcoptes scabiei) are of particular concern to humans. Ectoparasites of the order copepod crustaceans (sea lice), like Lepeoptheirus spp and Caligus spp (especially Lepeoptheirus salmonis and Caligus elongatus) are of particular concern for farmed fish. The desert locust and the beetle Meligethes aeneus are especially important pests for crop plants.

Typically, one seeks to eliminate such exoskeletal pests using organophosphates or pyrethroids. However, the pest species has an annoying tendency to become resistant to the organophosphate or pyrethroid being used and it becomes necessary to cycle between different biocides. Moreover such biocides, the organophosphates in particular, have been found to have detrimental side effects on higher life forms, in particular birds and humans, for example the appliers of the biocide or the consumers of the treated product or the treated pests. Since pyrethroids are less effective in warm climates, there is a tendency amongst farmers in warm regions to choose organophosphate biocides and as a result not only are their plant products so contaminated as to be unexportable to profitable markets but the pesticidal effect is diminished due to the side effects on the natural predators of the pest species, e.g. birds, reptiles, etc.

There have been proposals to treat simultaneously with a combination of a pyrethroid and an organophosphate or to treat first with one and then with the other so as to enhance the pesticidal efficiency. In general however such proposals involve the use of standard or only slightly below standard doses of both of the two pesticides and as a result the problems of organophosphates contamination or poisoning are barely reduced.

We have now found however that a combination, i.e. simultaneous, treatment with a pyrethroid and an organophosphate is surprisingly effective and involves little or no organophosphate contamination if the pyrethroid is administered at 10 to 120%, particularly 15 to 100%, especially 20 to 75%, of the normal dose and the organophosphate is administered at 0.05 to 3.5%, especially 0.1 to 3.0%, particularly 0.5 to 2.5% of the normal dose.

Thus viewed from one aspect the invention provides a method of pesticidal treatment of vertebrate aquatic animals, in particular fish, which method comprises applying to said aquatic animals, typically topically or, more preferably, by administration into water in which said aquatic animals are contained, a topical pesticidal composition comprising a pyrethroid and an organophosphate biocide in a weight ratio of 1:20 to 10:1, particularly 1:10 to 5:1, especially 1:3 to 3:1.

In place of pyrethroids, pyrethrins may be used and the term “pyrethroid” as used herein should be understood to include pyrethrins.

Viewed from a further aspect the invention provides a method of pesticidal treatment of plant crops, especially citrus fruit crops, which method comprises applying a composition to crop plants or soil seeded with crop plants a topical pesticidal composition comprising a pyrethroid and an organophosphate biocide in a weight ratio of 1:20 to 10:1, particularly 1:10 to 5:1, especially 1:3 to 3:1.

Viewed from a still further aspect the invention provides a method of pesticidal treatment of birds or vertebrate land animals which method comprises applying a composition comprising a pyrethroid and an organophosphate biocide in a weight ratio of 1:20 to 10:1, particularly 1:10 to 5:1, especially 1:3 to 3:1, to said birds or land animals, typically topically.

Viewed from a still further aspect the invention provides a topical marine pesticidal composition comprising a pyrethroid and an organophosphate in a weight ratio of 1:20 to 10:1, particularly 1:10 to 5:1, especially 1:3 to 3:1.

Viewed from another aspect the invention provides a topical pesticidal composition comprising (a) a pyrethroid selected from cypermethrin, deltamethrin, resmethrin, cyfluthrin, tau-fluvalinate, and fluvalinate, especially cypermethrin or deltamethrin, and (b) an organophosphate biocide in a weight ratio of 1:20 to 10:1, particularly 1:10 to 5:1, especially 1:3 to 3:1.

Particularly preferably, animals treated with the compositions according to the invention are also treated with a CYP enzyme inhibitor as a synergist for the pyrethroid/pyrethrin, e.g. piperonyl butoxide. Especially preferably said animals are treated with a mixed function oxidase (MFO) inhibitor. This may be present in the compositions according to the invention or alternatively may be administered with the animal's feed, e.g. 12 hours to 60 hours, especially 24 to 48 hours, before exposure to the compositions of the invention.

Insofar as crop plants are concerned, the method of the invention is especially applicable to plants for which the fruit or seeds are foodstuffs for animals, e.g. humans, more especially cereals, sugar cane, oil seed plants (e.g. Brassica napus), and particularly citrus. Moreover the method of invention is especially suited for performance between latitudes 45° N and 40° S, especially 40° N and 30° S, more especially 40° N and 20° N/20° S. For crop or field treatment, the pyrethroid dosage is preferably 2 to 50 g/ha, especially 5 to 25 g/ha, particularly 10 to 20 g/ha. The organophosphate dosage is preferably 1 to 50 g/ha, especially 2 to 25 g/ha, particularly 5 to 15 g/ha.

Insofar as land animals are concerned, the method of the invention is especially applicable to hair-bearing animals, particularly humans and farmed animals. The method may be used with indoor pets and woodland animals, such as dogs, cats and deer, but this is not particularly preferred as their infesting pests may spend much of their life cycle on the ground or in trees and thus may not be on the animal at the time of treatment. For the treatment of animals or birds, the pyrethroid concentration is preferably 1 to 200 ppm (by wt), especially 2 to 125 ppm, particularly 5 to 100 ppm. The organophosphate concentration is preferably 1 to 25 ppm, especially 3 to 20 ppm, particularly 6 to 15 ppm.

Insofar as aquatic animals are concerned, the method of the invention is particularly applicable to farmed, vertebrate fish, e.g. salmon (and other salmonids), cod, etc. Farmed salmon in particular are especially prone to infestation by sea lice. For the treatment of fish, the pyrethroid concentration is preferably 0.3 to 30 ppb (by wt), especially 0.5 to 20 ppb, particularly 1 to 15 ppb. The organophosphate concentration is preferably 0.5 to 20 ppb, especially 1 to 15 ppb, particularly 2 to 8 ppb. The preferred concentration for deltamethrin is 2 ppb, while that for cypermethrin is 5-15 ppb.

The organophosphate biocide of the methods, uses and compositions of the invention may be any of the conventional organophosphate biocides, for example malathion, parathion, dichlorvos, azamethiphos, chlorpyrifos, chlorthion, trichlorphon, methyl parathion, azinphos methyl, tetrachlorvinphos, phosmet, diazinon, coumaphos, bromophos, dioxathion, ethion, fenitrothion, acephate, triazophos, dicrotophos, isophenphos and fenchlorphos. Preferred organophosphate biocides include parathion, dichlorvos, azamethiphos, chlorthion, trichlorphon, methyl parathion, azinphos methyl, tetrachlorvinphos, phosmet, diazinon, coumaphos, bromophos, dioxathion, dicrotophos, isophenphos and fenchlorphos. The use of azamethiphos or dichlorvos however is preferred.

The pyrethroid biocide of the methods, uses and compositions of the invention may be any of the conventional pyrethroid biocides, for example permethrin (including cis- and trans-forms), phenothrin, fenvalerate, cypermethrin (including alpha- and zeta-cypermethrin), pyrethrin, bifenthrin, resmethrin, sumithrin, tetramethrin, cyfluthrin, transfluthrin, imiprothrin, tau-fluvalinate, fluvalinate, fenpropathrin and deltamethrin (also known as decamethrin). Particularly preferably the pyrethroid is selected from cypermethrin, pyrethrin, bifenthrin, sumithrin, tetramethrin, cyfluthrin, transfluthrin, imiprothrin, tau-fluvalinate, fluvalinate, fenpropathrin and deltamethrin, especially preferably it is selected from cypermethrin, deltamethrin, resmethrin, cyfluthrin, tau-fluvalinate, and fluvalinate. The use of deltamethrin or cypermethrin however is preferred.

Particularly preferred organophosphate and pyrethroid combinations include: azametiphos and deltamethrin; azametiphos and cypermethrin; fenitrothion and deltamethrin and azametiphos and tau-fluvalinate.

In some embodiments, the following pyrethroid/organophosphate combinations are less preferred: phenothrin and malathion; cypermethrin and ethion; cypermethrin and acephate; cypermethrin and chlorpyrifos; resmethrin and fenitrothion; deltamethrin and triazophos; deltamethrin and fenitrothion. In some aspects, less preferred pyrethroids are phenothrin, permethrin and fenvalerate.

The compositions of the invention will typically also include a solvent, for example water, an alcohol (e.g. a C₁₋₆ alcohol), or a water/alcohol mixture. If desired, a surfactant, a polyol or an oil may also be included to facilitate wetting or adhesion.

The concentration of the biocides in the compositions of the invention will depend on the manner of application—the more direct the mode of application, the higher the concentration can be as a general rule. Application typically will be by topical application to the surface of the host species, by administration into water containing the host species, or by spraying either the host species or a zone containing the host species, e.g. a field or orchard.

For topical treatment of human parasites, and for the treatment of lice in particular, the pyrethroid is preferably present in the composition at a concentration of 0.2 to 3% wt, especially 0.5 to 2% wt, particularly about 1% wt. The remaining components of the composition may be conventional components for topical compositions and may be present in conventional amounts, e.g. water, alcohol, gelling agents, surfactants, fragrances, etc.

The method of the invention may if necessary be repeated, e.g. after 7 to 10 days, but for a single case of infestation a single performance of the method will generally be sufficient.

Contact with the compositions of the invention is desirably maintained for 15 to 180 minutes at least, preferably 30 to 90 minutes, especially about 60 minutes.

The compositions according to the invention may advantageously contain a further biocide, e.g. selected from the chloronicotinyl (e.g. imidacloprid), phenylpyrazole (e.g. fipronil), oxadiazine (e.g. indoxacarb), pyrazole (e.g. chlorfenapyr), or organochlorine (e.g. lindane) classes. The phenylpyrazoles and organochlorines however are not preferred.

The compositions according to the invention may take any convenient topical application form, e.g. solution, dispersion, powder, etc. Since they will generally be diluted on or before application, their concentrations and formulations are not critical. Commercially available compositions may be admixed for use according to the methods and uses of the invention.

The compositions comprising a pyrethroid and an organophosphate described herein for use in therapy or medicine and for use in the methods herein described also form part of the invention. Viewed from a further aspect, the present invention therefore provides the use of a pyrethroid and an organophosphate in the manufacture of a medicament, preferably a topical medicament, for application to vertebrate aquatic animals, e.g. fish, or birds or vertebrate land animals to combat infestation by exoskeletal ectoparasites wherein the weight ratio of pyrethroid to organophosphate biocide is 1:20 to 10:1, particularly 1:10 to 5:1, especially 1:3 to 3:1.

The compositions as herein described for use in the methods of combating pests or pesticidal treatment of vertebrate aquatic animals (e.g. fish), bird and/or vertebrate land animals as herein described form a further embodiment of the invention.

From a further aspect, the present invention provides the use of a topical pesticidal composition comprising a pyrethroid and an organophosphate biocide in a weight ratio of 1:20 to 10:1, particularly 1:10 to 5:1, especially 1:3 to 3:1 in the pesticidal treatment of plant crops, especially citrus fruit crops.

For plant crops, the pest species treated will preferably be one resident on the plant crop rather than one, such as grasshoppers or locusts, which moves from plant to ground to plant and so on.

In place of, or partially in place of the organophosphate, a carbamate biocide may be used. Such compositions, uses and methods form further aspects of the present invention. One typical example of a carbamate biocide is carbaryl. The carbamates may be used in the dosages and ratios set out herein for organophosphates.

The invention will now be described with reference to the following non-limiting Examples.

### EXAMPLE 1

### Composition for Treatment of Salmon

A combination is made of 5 parts wt. azametiphos and 5 or 15 parts wt. of cypermethrin (or 2 or 3 parts wt. deltamethrin) and 1 billion parts wt. aqueous carrier solution. The lower pyrethroid dose may be used for non-pyrethroid-resistant sea-lice. Application may be by immersion of the fish in the composition, e.g. for around 80 minutes, for example 30 to 70 minutes.

### EXAMPLE 2

### Composition for Treatment of Citrus Crops

The compositions of Example 1 may be used, applied by spraying, e.g. to citrus trees.

### EXAMPLE 3

### Composition for the Treatment of Hair-Bearing Land Animal

Azametiphos (in concentrated solution in isopropanol) is added at 0.5% wt and deltamethrin is added at 1% wt to a commercially available shampoo, e.g. Head & Shoulders from Proctor & Gamble, or cream rinse/conditioner. After application, rinsing should be delayed for 30 to 60 minutes. Treatment is desirably effected twice in one day.

1. A topical pesticide composition for use in a method of pesticidal treatment of vertebrate aquatic animals, in particular fish, which method comprises applying said composition to said aquatic animals, typically topically or, more preferably, by administration into water in which said aquatic animals are contained, wherein said composition comprises a pyrethroid and an organophosphate biocide in a weight ratio of 1:20 to 10:1.

2. The composition as claimed in claim 1 for use in the pesticidal treatment of fish to combat infestation by ectoparasites, e.g. Lepeoptheirus salmonis or Caligus elongatus.

3. The composition as claimed in claim 1 wherein said weight ratio is 1:10 to 5:1.

4. The composition as claimed in claim 1 wherein said weight ratio is 1:3 to 3:1.

5. The composition as claimed in claim 1 wherein said pyrethroid is selected from permethrin, phenothrin, fenvalerate, cypermethrin, pyrethrin, bifenthrin, resmethrin, sumithrin, tetramethrin, cyfluthrin, transfluthrin, imiprothrin, tau-fluvalinate, fluvalinate, fenpropathrin and deltamethrin.

6. The composition as claimed in claim 1 wherein said organophosphate is selected from malathion, parathion, dichlorvos, azamethiphos, chlorpyrifos, chlorthion, trichlorphon, methyl parathion, azinphos methyl, tetrachlorvinphos, phosmet, diazinon, coumaphos, bromophos, dioxathion, ethion, fenitrothion, acephate, triazophos, dicrotophos, isophenphos and fenchlorphos.

7. A topical pesticidal composition comprising (a) a pyrethroid selected from cypermethrin, deltamethrin, resmethrin, cyfluthrin, tau-fluvalinate, and fluvalinate, especially cypermethrin or deltamethrin, and (b) an organophosphate biocide in a weight ratio of 1:20 to 10:1, particularly 1:10 to 5:1, especially 1:3 to 3:1.

8. The composition as claimed in claim 7 wherein said organophosphate biocide is azametiphos.

9. The composition as claimed in claim 7 for use in a method of pesticidal treatment of vertebrate aquatic animals, in particular fish, which method comprises applying said composition to said aquatic animals, typically topically or, more preferably, by administration into water in which said aquatic animals are contained.

10. A method of pesticidal treatment of vertebrate aquatic animals, in particular fish, which method comprises applying to said aquatic animals, typically topically or, more preferably, by administration into water in which said aquatic animals are contained, a topical pesticidal composition comprising a pyrethroid and an organophosphate biocide in a weight ratio of 1:20 to 10:1, particularly 1:10 to 5:1, especially 1:3 to 3:1.

11. A method of pesticidal treatment of plant crops, especially citrus fruit crops, which method comprises applying a composition to crop plants or soil seeded with crop plants a topical pesticidal composition comprising a pyrethroid and an organophosphate biocide in a weight ratio of 1:20 to 10:1, particularly 1:10 to 5:1, especially 1:3 to 3:1.

12. The method as claimed in claim 11 wherein said pesticidal treatment is to combat a pest which is resident on the plant crop.

13. The method as claimed in claim 10 wherein said pyrethroid is selected from permethrin, phenothrin, fenvalerate, cypermethrin, pyrethrin, bifenthrin, resmethrin, sumithrin, tetramethrin, cyfluthrin, transfluthrin, imiprothrin, tau-fluvalinate, fluvalinate, fenpropathrin and deltamethrin.

14. The method as claimed in claim 10 wherein said organophosphate is selected from malathion, parathion, dichlorvos, azamethiphos, chlorpyrifos, chlorthion, trichlorphon, methyl parathion, azinphos methyl, tetrachlorvinphos, phosmet, diazinon, coumaphos, bromophos, dioxathion, ethion, fenitrothion, acephate, triazophos, dicrotophos, isophenphos and fenchlorphos.

15. The method as claimed in claim 11 wherein said pyrethroid is selected from permethrin, phenothrin, fenvalerate, cypermethrin, pyrethrin, bifenthrin, resmethrin, sumithrin, tetramethrin, cyfluthrin, transfluthrin, imiprothrin, tau-fluvalinate, fluvalinate, fenpropathrin and deltamethrin.

16. The method as claimed in claim 11 wherein said organophosphate is selected from malathion, parathion, dichlorvos, azamethiphos, chlorpyrifos, chlorthion, trichlorphon, methyl parathion, azinphos methyl, tetrachlorvinphos, phosmet, diazinon, coumaphos, bromophos, dioxathion, ethion, fenitrothion, acephate, triazophos, dicrotophos, isophenphos and fenchlorphos.
